(19) United States
(12) Patent Application Publication
(10) Pub. No.: US 2019/0248549 A1
(43) Pub. Date: Aug. 15, 2019

(54) BREAST MILK STORAGE BAG

(71) Applicant: Xiamen Threestone Packing Material Co., Ltd., Xiamen (CN)

(72) Inventor: Rongkang Chen, Xiamen (CN)

(21) Appl. No.: 15/985,663

(22) Filed: May 21, 2018

(30) Foreign Application Priority Data
Feb. 11, 2018 (CN) 201820245525.5

(51) Int. Cl. B65D 33/25

(52) U.S. Cl. CPC B65D 33/2541; A61M 1/062

(57) ABSTRACT

A breast milk storage bag has a bag body. The bag body has an opening with a first broken line and a hanging portion below the opening. One of an upper part and a lower part of the hanging portion is provided with a broken line, and the other part is integrally formed with the bag body. This can ensure the use of the breast milk storage bag. When the milk is poured into the breast milk storage bag, the milk won't splash from the hanging portion to the outside of the bag body. The film of the hanging portion does not fall off, and the situation that the film falls into the bag body can be avoided.

### BACKGROUND OF THE INVENTION

### 1. Field of the Invention

The present invention relates to a bag, and more particularly to a breast milk storage bag.

### 2. Description of the Prior Art

Many breast-feeding women need to extract milk from the breasts for storage due to work and other reasons. Therefore, breast pumps and breast milk storage bags are widely used. In order to ensure hygiene, the use of such storage bags is mostly disposable.

As shown in FIG. 1, a conventional breast milk storage bag has a bag body 10. The periphery of the bag body 10 is compressed with high frequency heating to form a sealing edge 20. Opposite sides of the top end of the sealing edge 20 have notches 30, respectively. The notches 30 are convenient for a user to tear one end of the breast milk storage bag 1 to form an opening 40. A circular or rectangular hanging portion 50 is arranged below the opening 40. A broken line 60 is arranged around the hanging portion 50. When in use, the broken line 60 can be torn for the hanging portion 50 to form a hanging hole, so that the bag body 10 can be carried or the bag body 10 can be hung through the hanging hole. At least one zipper 70 is provided below the hanging portion 50. The zipper 70 can be opened to contain breast milk, and then the zipper 70 is closed to store the breast milk.

For the conventional disposable breast milk storage bag, the broken line 60 is arranged around the hanging portion 50. When the hanging portion 50 is to be used, the broken line 60 is torn and the two films on the hanging portion 50 will fall. At this time, if the zipper 70 is in an open state, the dropped film may fall into the bag body 10 to result in a certain hygiene risk. In addition, after tearing the hanging portion 50, when the breast milk is poured from the opening 40 into the bag body 10, the breast milk may flow out from the hanging hole to cause a waste. Furthermore, the conventional breast milk storage bag does not have any guide design for tearing. The tearing of the bag body 10 depends only on the notches 30 of the sealing edge 20. The strong tearing tends to have a result that both sides of the opening 40 have inconsistent tearing sections. When the tearing is performed, the tearing section may extend to the position of the zipper 70, resulting in scrapping of the breast milk storage bag.

Accordingly, the inventor of the present invention has devoted himself based on his many years of practical experiences to solve these problems.

### SUMMARY OF THE INVENTION

The primary object of the present invention is to provide a breast milk storage bag that is more resistant to leakage when breast milk is poured in, and can be used more sanitarily.

In order to achieve the aforesaid object, the breast milk storage bag of the present invention has a bag body. The bag body has an opening with a first broken line at its top end, a hanging portion below the opening, and at least one zipper below the hanging portion. One of an upper part and a lower part of the hanging portion is provided with a second broken line, and the other part is integrally formed with the bag body.

Preferably, the hanging portion has a rectangular shape. The second broken line of the hanging portion is a U-shaped configuration with an opening facing upwards or downwards.

Preferably, the hanging portion has a circular shape. The second broken line of the hanging portion is a semicircular configuration.

Preferably, the first broken line of the opening of the bag body has a convex semicircle shape.

Preferably, the at least one zipper includes two zippers.

Preferably, the bag body is provided with a remark tag between the opening and the hanging portion.

Preferably, the bag body is provided with a volume scale mark thereon.

According to the above structure, the breast milk storage bag of the present invention has the hanging portion designed as a partially broken structure, so that the hanging portion can create a hanging hole without separating from the bag body to ensure the use of the breast milk storage bag. When the milk is poured into the breast milk storage bag, the milk won't splash from the hanging portion to the outside of the bag body. The film of the hanging portion does not fall off, and the situation that the film falls into the bag body can be avoided, preventing infants and young children from eating inadvertently to resulting in suffocation, so the use is more hygienic and safe.

### BRIEF DESCRIPTION OF THE DRAWINGS

FIG. 1 is a schematic view of a conventional breast milk storage bag; and

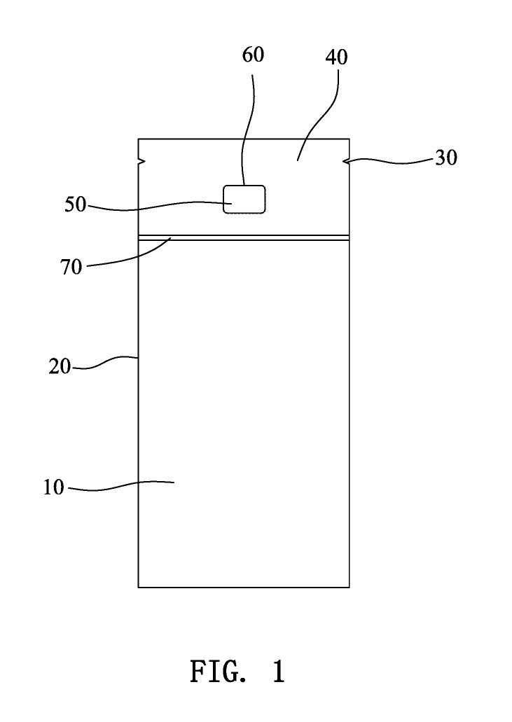

FIG. 2 is a schematic view of a breast milk storage bag in accordance with the present invention.

### DETAILED DESCRIPTION OF THE PREFERRED EMBODIMENTS

Embodiments of the present invention will now be described, by way of example only, with reference to the accompanying drawings.

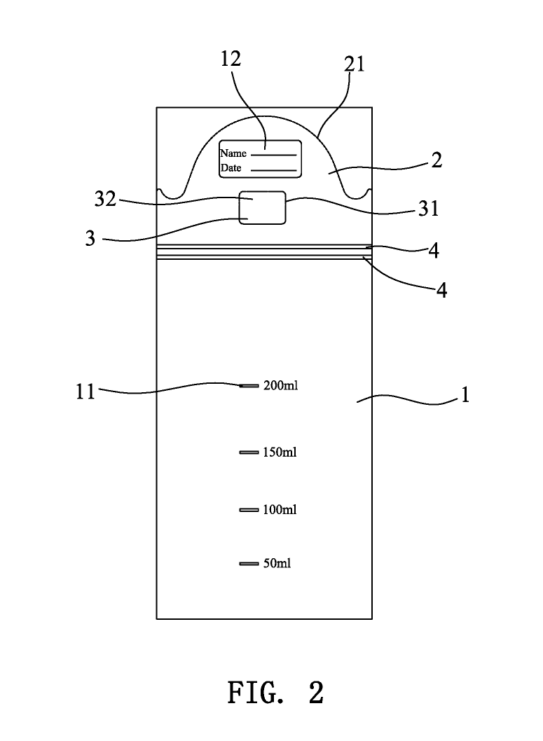

As shown in FIG. 2, the present invention discloses a breast milk storage bag. The breast milk storage bag has a bag body 1. The bag body 1 has an opening 2 with a first broken line 21 at its top end, a hanging portion 3 below the opening 2, and at least one zipper 4 below the hanging portion 3. In this embodiment, the main body 1 has two zippers 4. The milk in the bag body 1 can be sealed by sealing the zippers 4, and the two zippers 4 can ensure the sealing property of the bag body 1. One of an upper part and a lower part of the hanging portion 3 is provided with a second broken line 31, and the other part is integrally formed with the bag body 1, that is, the other part is not provided with the second broken line 31. When the broken line 31 is torn, a hanging hole 32 will be formed, and the hanging portion 3 will not be separated from the bag body 1, so that the bag body 1 can be hung through the hanging hole 32 of the hanging portion 3 or can be carried by hand. The hanging portion 3 won't be separated from the bag body 1, which can prevent the hanging portion 3 from falling into the bag body 1. When the milk is poured, the milk does not flow out of the bag body 1 from the hanging hole.

The structure of the hanging portion 3 can be designed in various shapes. For example, the hanging portion may be in a rectangular shape, and the second broken line 31 of the hanging portion 3 may be a U-shaped configuration with an opening facing upwards or downwards. The hanging portion may be in a circular shape. When the hanging portion has a circular shape, the second broken line 31 of the hanging portion 3 is a semicircular configuration. When the second broken line 31 is torn, a semicircular hanging hole 32 is formed.

In order to facilitate the opening of the bag body 1 and pouring the milk, the first broken line 21 of the opening 2 of the bag body 1 may be in a convex semicircle shape.

The bag body 1 may be provided with a volume scale mark 11 for observing the amount of poured milk and the amount of milk consumed by the baby.

The bag body 1 may be provided with a remark tag 12 between the opening 2 and the hanging portion 3 to note the date or other notes.

When in use, the bag body 1 is torn along the first broken line 21 of the opening 2, the breast milk is poured into the bag body, the zippers 4 are pressed tightly, and then the bag body is placed in the refrigerator to refrigerate or freeze the breast milk. When it is necessary to use the breast milk, the zippers 4 are opened, the breast milk is poured into a bottle and then heated for the baby to eat; or the bag body 1 may be directly put into hot water and then the breast milk is poured into a bottle.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A breast milk storage bag, having a bag body, the bag body having an opening with a first broken line at its top end, a hanging portion below the opening and at least one zipper below the hanging portion, characterized in that one of an upper part and a lower part of the hanging portion is provided with a second broken line, and the other part is integrally formed with the bag body.

2. The breast milk storage bag as claimed in claim 1, wherein the hanging portion has a rectangular shape, and the second broken line of the hanging portion is a U-shaped configuration with an opening facing upwards or downwards.

3. The breast milk storage bag as claimed in claim 1, wherein the hanging portion has a circular shape, and the second broken line of the hanging portion is a semicircular configuration.

4. The breast milk storage bag as claimed in claim 1, wherein the first broken line of the opening of the bag body has a convex semicircle shape.

5. The breast milk storage bag as claimed in claim 1, wherein the at least one zipper includes two zippers.

6. The breast milk storage bag as claimed in claim 1, wherein the bag body is provided with a remark tag between the opening and the hanging portion.

7. The breast milk storage bag as claimed in claim 1, wherein the bag body is provided with a volume scale mark thereon.
